(19) United States
(12) Patent Application Publication
(10) Pub. No.: US 2024/0050772 A1
(43) Pub. Date: Feb. 15, 2024

(54) MEDICAL ULTRASOUND INCISION DEVICE AND METHOD OF MANUFACTURING MEDICAL ULTRASOUND INCISION DEVICE

(71) Applicant: Olympus Corporation, Tokyo (JP)

(72) Inventor: Kohei TOMITA, Tokyo (JP)

(73) Assignee: Olympus Corporation, Tokyo (JP)

(21) Appl. No.: 17/884,166

(22) Filed: Aug. 9, 2022

(51) Int. Cl. A61N 7/00

(52) U.S. Cl. CPC A61N 7/00; A61B 2090/378

(57) ABSTRACT

A medical ultrasound incision device includes: a probe configured to vibrate ultrasonically; a jaw configured to be opened and closed relatively to the probe and hold a body tissue between the probe and the jaw; and a pad that is positioned between the probe and the jaw, the pad being configured to prevent direct contact between the probe and the jaw in a state where the jaw has been closed, the pad including polyethylene having molecular weight larger than 4,400,000.

### BACKGROUND

### 1. Technical Field

The present disclosure relates to a medical ultrasound incision device and a method of manufacturing the medical ultrasound incision device.

### 2. Related Art

Medical ultrasound incision devices, which are for treatment of body tissue by use of energy, such as ultrasound vibration, have been known as devices for minimally invasive surgical operations. Examples of this treatment include incision and coagulation.

A medical ultrasound device includes a probe that vibrates ultrasonically and a jaw that is capable of: being opened and closed with respect to the probe; and holding, together with the probe, a body tissue. A pad made of a resin material high in sliding performance, such as a fluorine-based resin, is arranged between the probe and the jaw (see, for example, Japanese Patent No. 6211228). This pad that comes into contact with the vibrating probe becomes worn. A medical ultrasound incision device having a pad with improved resistance to wear has been proposed (for example, Japanese Patent No. 5539239). This pad includes a first region and a second region including materials different from each other and is elastically flexible along a direction vertical to a clamping surface.

There is a great demand for a pad having improved resistance to wear for medical ultrasound incision devices.

### SUMMARY

In some embodiments, a medical ultrasound incision device includes: a probe configured to vibrate ultrasonically; a jaw configured to be opened and closed relatively to the probe and hold a body tissue between the probe and the jaw; and a pad that is positioned between the probe and the jaw, the pad being configured to prevent direct contact between the probe and the jaw in a state where the jaw has been closed, the pad including polyethylene having molecular weight larger than 4,400,000.

In some embodiments, provided is a method of manufacturing a medical ultrasound incision device comprising a probe vibrating ultrasonically. The method includes: attaching a pad to a jaw, the jaw being configured to be opened and closed relatively to the probe and hold a body tissue between the probe and the jaw, the pad being configured to prevent direct contact between the probe and the jaw in a state where the jaw has been closed, the pad including polyethylene having molecular weight larger than 4,400,000.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of a medical ultrasound incision device according to an embodiment.

FIG. 2 is a sectional view of a transducer unit illustrated in FIG. 1.

FIG. 3 is a perspective view of a jaw and a distal end portion of a probe of a handpiece that are illustrated in FIG. 1.

FIG. 4 is a graph illustrating molecular weight of polyethylene and results of evaluation of durability against ultrasound.

FIG. 5 is a graph illustrating a relation between molecular weight of polyethylene and durability against ultrasound, the relation being calculated on the basis of the results in FIG. 4.

### DETAILED DESCRIPTION

By reference to the appended drawings, embodiments will be described hereinafter.

### Embodiments

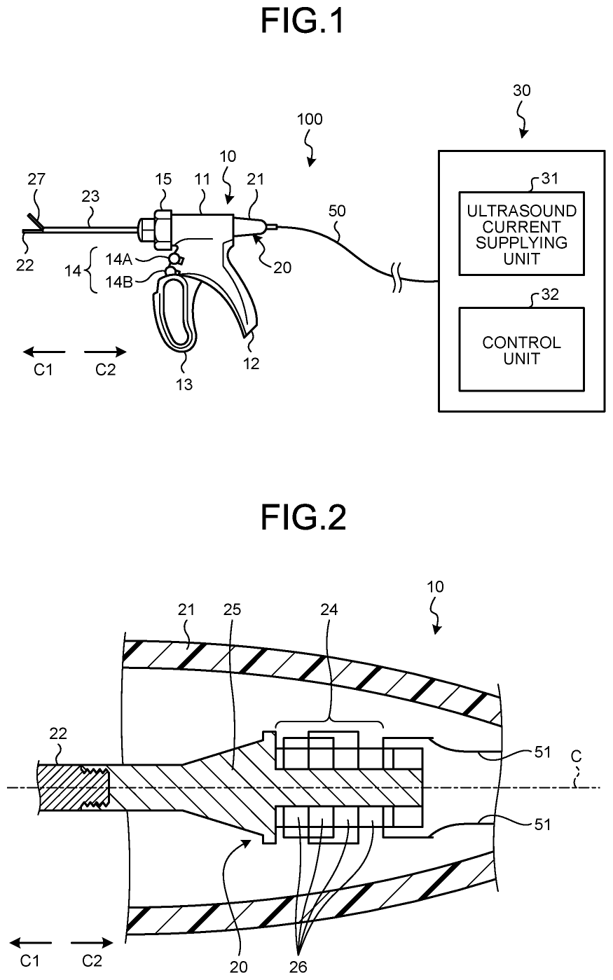

FIG. 1 is a diagram illustrating a schematic configuration of a medical ultrasound incision device according to an embodiment. A medical ultrasound incision device 100 is a treatment tool for operation, such as incision and coagulation, of a body tissue to be treated by application of energy from ultrasound vibration to the body tissue. The medical ultrasound incisions device 100 may apply high frequency energy, in addition to the energy from ultrasound vibration. The medical ultrasound incision device 100 has a longitudinal axis C. A direction indicated by an arrow C1 in FIG. 1 is a distal direction along the longitudinal axis C and a direction indicated by an arrow C2 in FIG. 1 is a proximal direction opposite to the distal direction.

The medical ultrasound incision device 100 includes a handpiece 10, a power source unit 30, and a cable 50 that connects the handpiece 10 and the power source unit 30 to each other.

The handpiece 10 includes a housing 11 that is a casing, a fixed handle 12 provided integrally with the housing 11, and a movable handle 13 that is capable of moving around. The fixed handle 12 includes an operation button 14 that is an energy operation input unit. The handpiece 10 also includes a rotating operation knob 15 at a distal end of the housing 11. The rotating operation knob 15 is rotatable about the longitudinal axis C, relatively to the housing 11.

The handpiece 10 includes a transducer unit 20. The transducer unit 20 is housed in a case 21 attachable to and detachable from the housing 11.

FIG. 2 is a sectional view of the transducer unit 20 illustrated in FIG. 1. The transducer unit 20 includes an ultrasound transducer 24 and a horn member 25. The ultrasound transducer 24 includes plural piezoelectric elements 26 provided therein. The piezoelectric elements 26 change electric current into ultrasound vibration. One end of an electric wiring 51 is connected to the ultrasound transducer 24. The electric wiring 51 is provided in a cable 50 and has the other end connected to an ultrasound current supplying unit 31 in the power source unit 30. In response to electric power being supplied from the ultrasound current supplying unit 31 to the ultrasound transducer 24 via the electric wiring 51, the ultrasound transducer 24 generates ultrasound vibration.

The horn member 25 includes a sectional area changing portion that has an approximately conical shape that gradually decreases in sectional area in the distal direction. The ultrasound transducer 24 is installed at a portion of the horn member 25, the portion being further proximal to the sectional area changing portion. The ultrasound vibration generated by the ultrasound transducer 24 is increased in amplitude at the sectional area changing portion.

A probe 22 is connected to a distal end of the transducer unit 20 and the periphery of the probe 22 is covered with a sheath 23 that is cylindrically shaped. The probe 22 is formed of a biocompatible metallic material, such as a titanium alloy. In a state where the probe 22 has been connected to the horn member 25, ultrasound vibration is transmitted from the horn member 25 to the probe 22.

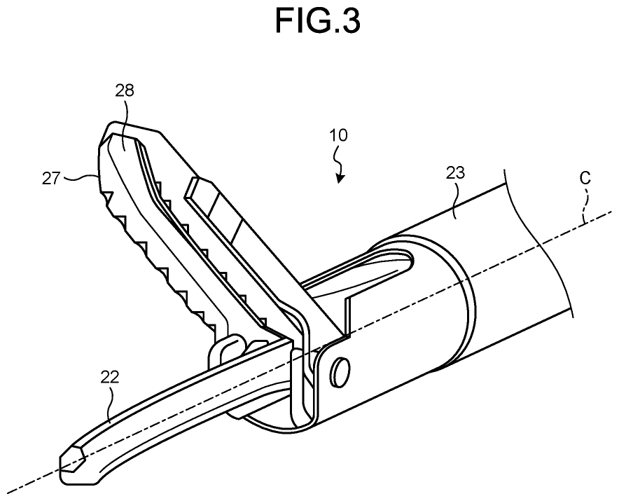

FIG. 3 is a perspective view of a jaw and a distal end portion of a probe of a handpiece, illustrated in FIG. 1. The sheath 23 is attached, at a proximal end of the sheath 23, to the housing 11, to be rotatable relatively to the housing 11. The rotating operation knob 15 is fixed to the sheath 23. A jaw 27 that is a holding unit is attached to a distal end of the sheath 23. The jaw 27 is capable of: being opened and closed relatively to the probe 22; and holding the body tissue by rotary movement of the jaw 27. A pad 28 is provided between the jaw 27 and the probe 22 to prevent direct contact of the jaw 27 and the probe 22.

One end of the cable 50 is connected to the case 21. Rotating the rotating operation knob 15 rotates the sheath 23, the probe 22, the horn member 25, the ultrasound transducer 24, and the jaw 27, all about the central axis C.

The power source unit 30 includes the ultrasound current supplying unit 31 and a control unit 32. The control unit 32 controls supply of ultrasound generating current from the ultrasound current supplying unit 31. In response to the operation button 14 being operated by an operating surgeon, an electric signal is transmitted to the control unit 32 and start of an energy operation is detected. The control unit 32 thereby supplies the ultrasound generating current from the ultrasound current supplying unit 31 to the transducer unit 20. The medical ultrasound incision device 100 illustrated in FIG. 1 enables incision and coagulation treatment by means of ultrasound vibration. In response to an operating surgeon operating a first operation button 14A, ultrasound energy and high frequency energy suitable for coagulation and incision of body tissue are output from the probe 22. In response to an operating surgeon operating a second operation button 14B, high frequency energy suitable for coagulation of body tissue is output from the probe 22. The medical ultrasound incision device 100 according to this embodiment enables incision and coagulation treatment through operation of the operation button 14. However, the medical ultrasound incision device 100 can be dedicated to incisions only.

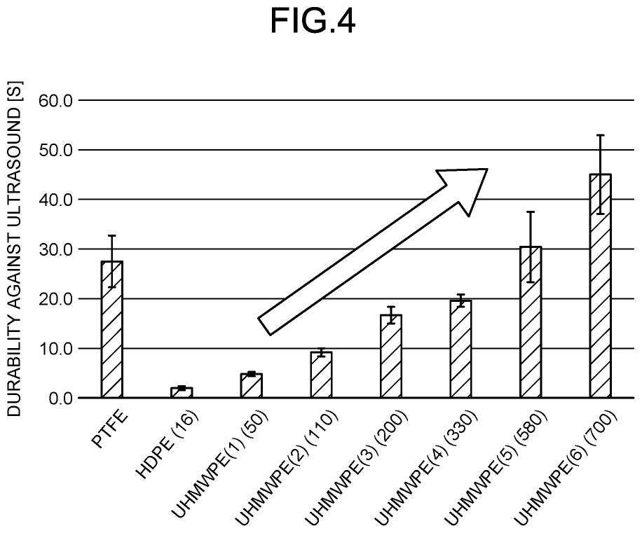

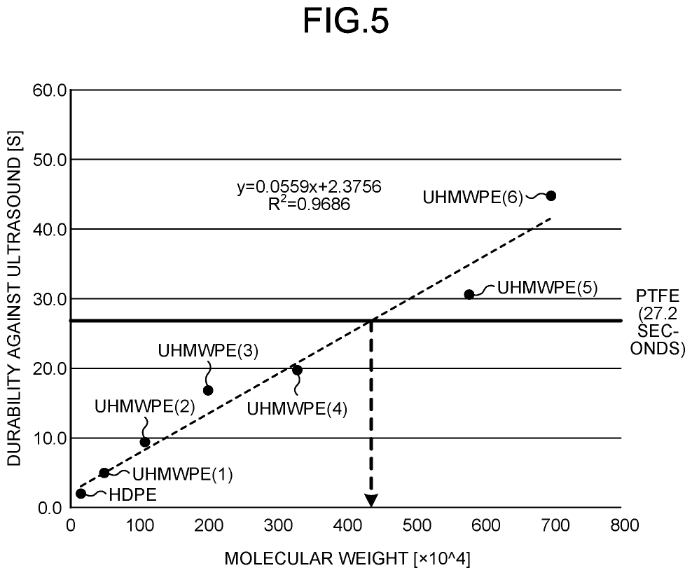

The pad 28 preferably to be attached to the jaw 27 will be described next. The pad 28 includes polyethylene having molecular weight larger than 4,400,000. Polyethylene having molecular weight larger than 4,400,000 is ultra high molecular weight polyethylene (UHMWPE) and preferably includes polyethylene having molecular weight of 5,800,000 or larger. FIG. 4 is a graph illustrating molecular weight of polyethylene and results of evaluation of durability against ultrasound, and FIG. 5 is a graph illustrating a relation between molecular weight of polyethylene and durability against ultrasound, the relation being calculated on the basis of the results in FIG. 4. Ultra high molecular weight polyethylene, high density polyethylene (HDPE), and polytetrafluoroethylene (PTFE) were each used as a pad, ultrasound oscillation was caused with the pad brought into direct contact with a probe, and time taken for the pad to be deformed (durability against ultrasound) was evaluated. PTFE has been adopted conventionally as a material for pads. Molecular weight of HDPE and UHMWPE in FIG. 4 is each 10,000 times the numerical value in parentheses. As illustrated in FIG. 4, it was confirmed that the higher the molecular weight of ultra high molecular weight polyethylene was, the more improved the durability against ultrasound was. Furthermore, as illustrated in FIG. 5, polyethylene having molecular weight larger than 4,400,000 is presumed to be higher in durability against ultrasound than PTFE is. Polyethylene having molecular weight of 5,800,000 or larger is high in durability against ultrasound and even more preferable. Molecular weight of ultra high molecular weight polyethylene is preferably large, but is preferably 20,000,000 or smaller in terms of cost and moldability.

Furthermore, a pad preferably includes 50% or more and 100% or less by mass of polyethylene having molecular weight larger than 4,400,000. Examples of a material that may be included in the pad in addition to polyethylene having molecular weight larger than 4,400,000 include: a resin material high in thermal resistance, such as polyetheretherketone (PEEK) or polytetrafluoroethylene; inorganic filler for radiation of heat; and a low molecular weight organic material used as a lubricant.

Because the pad including polyethylene having molecular weight larger than 4,400,000 is used in the medical ultrasound incision device according to the embodiment; deformation is not caused for a long period of time even if ultrasound oscillation is caused in a state where the probe and the pad are in direct contact with each other, and excellent durability is thus achieved.

In the embodiment described above, the pad including polyethylene having molecular weight larger than 4,400,000 is single-layered, but as long as the pad includes polyethylene having molecular weight larger than 4,400,000, the pad may be double-layered. For example, a pad that may be suitably used includes: a first layer including polyethylene having molecular weight larger than 4,400,000; and a second layer including polytetrafluoroethylene. In a case where a pad includes a layer including polyethylene having molecular weight larger than 4,400,000 and a layer not including polyethylene having molecular weight larger than 4,400,000; the layer not including polyethylene having molecular weight larger than 4,400,000, that is, the second layer including polytetrafluoroethylene in the above example, is preferably attached to the jaw and the layer including polyethylene having molecular weight larger than 4,400,000 is preferably brought into direct contact with the probe.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical ultrasound incision device, comprising:
a probe configured to vibrate ultrasonically;
a jaw configured to be opened and closed relatively to the probe and hold a body tissue between the probe and the jaw; and
a pad that is positioned between the probe and the jaw, the pad being configured to prevent direct contact between the probe and the jaw in a state where the jaw has been closed, the pad including polyethylene having molecular weight larger than 4,400,000.

2. The medical ultrasound incision device according to claim 1, wherein the molecular weight of the polyethylene included in the pad is 20,000,000 or smaller.

3. The medical ultrasound incision device according to claim 1, wherein the molecular weight of the polyethylene included in the pad is 5,800,000 or larger.

4. The medical ultrasound incision device according to claim 1, wherein the pad includes 50% or more and 100% or less by mass of the polyethylene.

5. The medical ultrasound incision device according to claim 1, further comprising:
a second pad including polytetrafluoroethylene, wherein the pad is attached to the jaw with the second pad interposed between the pad and the jaw.

6. A method of manufacturing a medical ultrasound incision device comprising a probe vibrating ultrasonically, the method comprising:
attaching a pad to a jaw, the jaw being configured to be opened and closed relatively to the probe and hold a body tissue between the probe and the jaw, the pad being configured to prevent direct contact between the probe and the jaw in a state where the jaw has been closed, the pad including polyethylene having molecular weight larger than 4,400,000.
